(19) United States
(12) Patent Application Publication
(10) Pub. No.: US 2019/0173683 A1
(43) Pub. Date: Jun. 6, 2019

(54) INFORMATION PROCESSING APPARATUS AND INFORMATION PROCESSING SYSTEM

(71) Applicant: FUJI XEROX CO., LTD., Tokyo (JP)

(72) Inventors: Minoru MITSUI, Kanagawa (JP); Katsunori KAWANO, Kanagawa (JP); Akinori KOMURA, Kanagawa (JP); Ryoji HORITA, Kanagawa (JP)

(73) Assignee: FUJI XEROX CO., LTD., Tokyo (JP)

(21) Appl. No.: 16/125,781

(22) Filed: Sep. 10, 2018

(30) Foreign Application Priority Data
Dec. 5, 2017 (JP) 2017-233630

(51) Int. Cl. H04L 12/18; A61B 5/11; A61B 5/0205

(52) U.S. Cl. CPC H04L 12/1822; G06K 9/00302; A61B 5/02055; A61B 5/1118

(57) ABSTRACT

An information processing apparatus includes a determining unit that determines an activity level of an attendee attending a meeting in accordance with an analysis based on biological information acquired from a body of the attendee, an instructing unit that provides instructions on a mode in which the attendee is to be represented in accordance with the activity level, a generating unit that generates information concerning the attendee represented in the mode, and a transmitter that transmits the generated information concerning the attendee.

### CROSS-REFERENCE TO RELATED APPLICATIONS

This application is based on and claims priority under 35 USC 119 from Japanese Patent Application No. 2017-233630 filed Dec. 5, 2017.

### BACKGROUND

### (i) Technical Field

The present disclosure relates to an information processing apparatus and an information processing system.

### (ii) Related Art

There are provided communication systems that enable attendees at remote locations to have a meeting, such as a conference, via a communication network. In such communication systems, limited information captured by using tools such as a camera and a microphone is used, and thus it is difficult to perceive a state of an attendee at a remote location, compared with a meeting in which attendees communicate with each other face-to-face. Therefore, techniques have been developed to use biological information obtained from bodies of attendees in addition to visual information and voice information in a conference or the like involving a remote location.

Japanese Unexamined Patent Application Publication No. 2003-16022 discloses a method of connecting remote locations to each other to have a conference in which exchange of text data between a plurality of computers is performed and a biological reaction of each attendee who uses one of the plurality of computers adapted for the conference is measured, so that a font used to display text data is varied in accordance with the measurement result of the biological reaction.

Japanese Unexamined Patent Application Publication No. 2016-91490 discloses a program for a conference system that includes a body measuring unit that measures biological information correlating with personal productivity and a stimulus providing unit that provides a plurality of attendees in a meeting with stimuli to improve the total productivity of the meeting.

Japanese Unexamined Patent Application Publication No. 2008-306586 discloses a method of deducing a state of an attendee at a remote location. This method uses a biological sensor to obtain a piece of biological data of a subject at a computer site on a network system in which a plurality of computer sites are connected to each other via a network, and pieces of biological data of attendees at the plurality of computer sites are processed or merged, so that the states of attendees at remote locations are able to be determined as if the attendees were in the immediate vicinity.

### SUMMARY

In a meeting held by connecting remote locations to each other, the eagerness of attendees at remote locations to participate in the meeting is difficult to perceive, compared with a case where attendees are present at a single location.

Aspects of a non-limiting embodiment of the present disclosure relate to conveying the eagerness of attendees at remote locations to participate in a meeting.

Aspects of a certain non-limiting embodiment of the present disclosure overcome the above disadvantages and/or other disadvantages not described above. However, aspects of the non-limiting embodiment are not required to overcome the disadvantages described above, and aspects of the non-limiting embodiment of the present disclosure may not overcome any of the disadvantages described above.

According to an aspect of the present disclosure, there is provided an information processing apparatus that includes a determining unit that determines an activity level of an attendee attending a meeting in accordance with an analysis based on biological information acquired from a body of the attendee, an instructing unit that provides instructions on a mode in which the attendee is to be represented in accordance with the activity level, a generating unit that generates information concerning the attendee represented in the mode, and a transmitter that transmits the generated information concerning the attendee.

### BRIEF DESCRIPTION OF THE DRAWINGS

An exemplary embodiment of the present disclosure will be described in detail based on the following figures, wherein:

FIG. 1 depicts a configuration of an information processing system according to an exemplary embodiment;

FIG. 2 depicts an example configuration of an information collecting apparatus;

FIG. 3 depicts an example configuration of a terminal;

FIG. 4 depicts a configuration of a server;

FIGS. 5A, 5B, 5C, and 5D depict databases stored in a memory;

FIG. 6 depicts a functional configuration of the server;

FIG. 7 is a flowchart depicting an operation flow performed by the server;

FIG. 8 depicts an example image displayed on the display of the terminal; and

FIG. 9 depicts an example image displayed on the display of the terminal in a modification.

### DETAILED DESCRIPTION

### Exemplary Embodiment

### Overall Configuration of Information Processing System

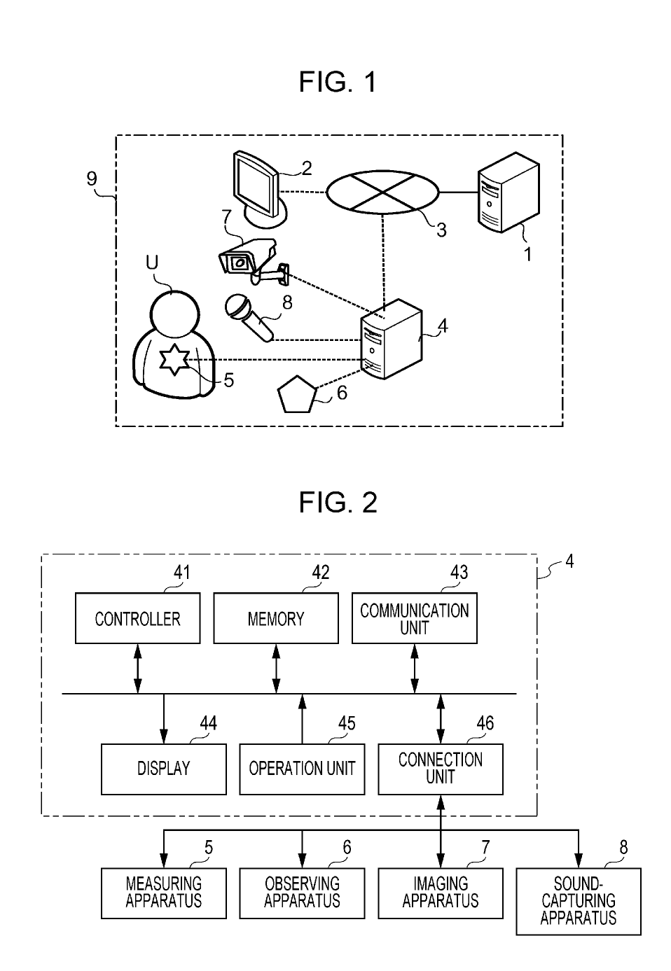

FIG. 1 depicts a configuration of an information processing system 9 according to an exemplary embodiment. The information processing system 9 includes a server 1, a terminal 2, an information collecting apparatus 4, and a communication network 3 via which these apparatuses are communicatively connected to each other. The information processing system 9 also includes a measuring apparatus 5, an observing apparatus 6, an imaging apparatus 7, and a sound-capturing apparatus 8. In the information processing system 9 depicted in FIG. 1, the measuring apparatus 5, the observing apparatus 6, the imaging apparatus 7, and the sound-capturing apparatus 8 are connected to the information collecting apparatus 4.

The information processing system 9 may include a plurality of servers 1 and a plurality of communication networks 3. In this exemplary embodiment, the communication network 3 connects a plurality of venues, and the terminal 2, the information collecting apparatus 4, the measuring apparatus 5, the observing apparatus 6, the imaging apparatus 7, and the sound-capturing apparatus 8 are disposed in each venue. To simplify descriptions, a single unit is depicted for each of these apparatuses in FIG. 1.

The server 1 is an information processing apparatus that provides information representing an attendee in a mode in accordance with the activity level of the attendee in a meeting held by connecting a plurality of venues to each other.

The term “meeting” indicates an event that is held by connecting a plurality of venues to each other via a communication network and that enables attendees present in each venue to communicate with each other through actions such as speaking and gesturing. Examples of such events include a conference, a debate, a study group, a party, and a game involving many participants.

The terminal 2 is an apparatus that provides users of the information processing system 9, such as attendees in the meeting or the facilitator of the meeting, with information concerning attendees. The communication network 3 communicatively connects the server 1, the terminal 2, and the information collecting apparatus 4 to each other and may be the Internet, for example.

At least one information collecting apparatus 4 is disposed, for example, in each venue and collects information concerning attendees U who come to the venue and attend a meeting. The information collecting apparatus 4 is connected to the measuring apparatus 5, the observing apparatus 6, the imaging apparatus 7, and the sound-capturing apparatus 8 and collects information provided by these apparatuses to send to the server 1.

In FIG. 1, the information collecting apparatus 4 is connected to each of the measuring apparatus 5, the observing apparatus 6, the imaging apparatus 7, and the sound-capturing apparatus 8 but may be connected to a plurality of any of these apparatuses. Alternatively, any of the observing apparatus 6, the imaging apparatus 7, and the sound-capturing apparatus 8 may be disconnected from the information collecting apparatus 4.

The measuring apparatus 5 is an apparatus that obtains information from a body of an attendee U (hereinafter, referred to as biological information), such as an electrocardiographic waveform, a pulse wave, and heart sounds of the attendee U. For example, the measuring apparatus 5 includes a plurality of electrodes to be attached to the skin of the attendee U and measures impedances between the plurality of electrodes to obtain an electrocardiographic waveform.

Biological information is information that changes depending on a mental state and includes an electrocardiogram, a variation in voice, facial expression, breathing rate, pulse wave, an electroencephalogram, a variation in cerebral blood flow, eye-fixation-related potential, amount of sweating, temperature change of peripheral skin, autonomic activity, change in the line of sight, pupil size, posture, and change in blood pressure.

The observing apparatus 6 is an apparatus that obtains information from the environment surrounding the attendee U (hereinafter, referred to as environmental information), such as temperature, humidity, wind direction, wind speed, odor, and vibration in a venue where the attendee U is present.

The imaging apparatus 7 is an apparatus that captures a facial expression and an appearance of the attendee U and generates image data representing an image thus captured. The imaging apparatus 7 includes an imaging device and an optical system including lenses and reflecting mirrors that are used for forming an optical image of an object on the optical receiving surface of the imaging device. Examples of the imaging device include a complementary metal-oxide semiconductor (CMOS) image sensor and a charge-coupled device (CCD) image sensor. The imaging apparatus 7 may be controlled by the information collecting apparatus 4 to adjust parameters such as the imaging area, the angle of view, and the focus.

The sound-capturing apparatus 8 is a microphone of a various type, such as a dynamic type or a condenser type, and captures the voice of the attendee U and the sound originating in the area surrounding the attendee U. Then the sound-capturing apparatus 8 generates audio data that represents the captured sound. The sound-capturing apparatus 8 may be controlled by the information collecting apparatus 4 to adjust the directivity.

### Configuration of Information Collecting Apparatus

FIG. 2 depicts an example configuration of the information collecting apparatus 4. The information collecting apparatus 4 includes a controller 41, a memory 42, a communication unit 43, a display 44, an operation unit 45, and a connection unit 46.

The controller 41 includes a central processing unit (CPU), a read-only memory (ROM), and a random-access memory (RAM). The CPU reads and executes computer programs (hereinafter, referred to simply as programs) to control each unit in the information collecting apparatus 4, and these programs are stored in the ROM and the memory 42.

The communication unit 43 is a communication circuit to connect to the communication network 3 wirelessly or by cable. The information collecting apparatus 4 exchanges information via the communication unit 43 with the server 1 connected to the communication network 3.

The operation unit 45, which includes input operation controls to provide various instructions, such as an operation button, a keyboard, and a touch panel, accepts an operation performed by a user and sends a signal corresponding to the operation to the controller 41.

The display 44 includes a display screen, such as a liquid crystal display, and displays an image in accordance with control by the controller 41. A transparent touch panel of the operation unit 45 may be superimposed on the display screen.

The information collecting apparatus 4 may optionally be constituted without the operation unit 45 or the display 44.

The memory 42 is a high-capacity memory, such as a solid-state drive or a hard disk drive, and stores various programs, data, and the like that are read by the CPU in the controller 41.

The connection unit 46 is an interface to be connected to apparatuses that provide various kinds of information. The connection unit 46 depicted in FIG. 2 is connected to the measuring apparatus 5, the observing apparatus 6, the imaging apparatus 7, and the sound-capturing apparatus 8.

The connection unit 46 obtains biological information concerning the attendee U, such as an electrocardiographic waveform, from the measuring apparatus 5 and environmental information concerning the attendee U from the observing apparatus 6.

In addition, the connection unit 46 obtains image data from the imaging apparatus 7 and audio data from the sound-capturing apparatus 8. The image data represents a captured image of an appearance of the attendee U, and the audio data represents the voice of the attendee U and the sound originating in the area surrounding the attendee U. Various kinds of information obtained by the connection unit 46 are provided to the controller 41, associated with the attendee U in accordance with control by the controller 41, and transmitted to the server 1 via the communication unit 43 and the communication network 3.

### Configuration of Terminal

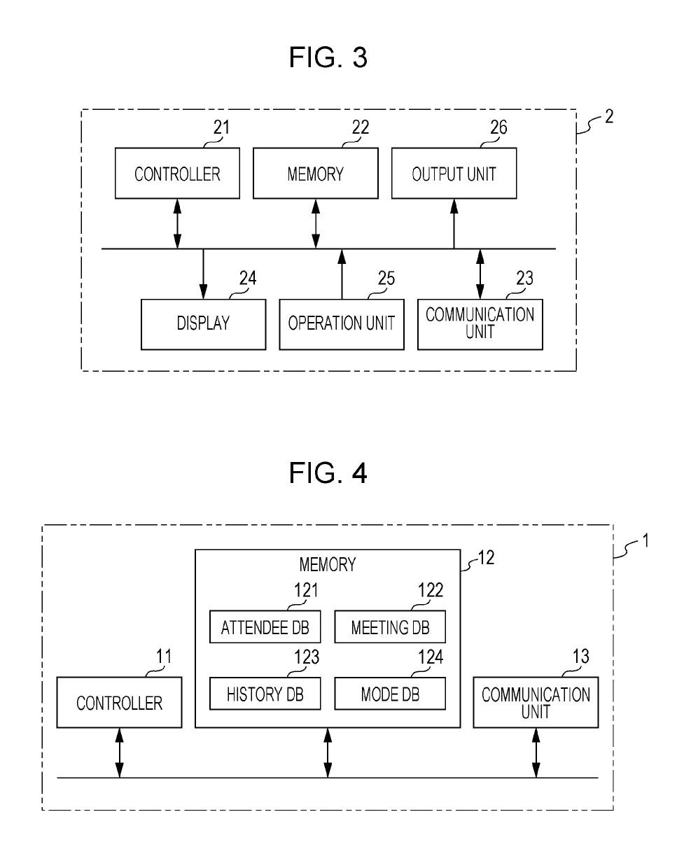

FIG. 3 depicts an example configuration of the terminal 2. The terminal 2 includes a controller 21, a memory 22, a communication unit 23, a display 24, an operation unit 25, and an output unit 26.

The controller 21 includes a CPU, a ROM, and a RAM. The CPU reads and executes programs to control each unit in the terminal 2, and these programs are stored in the ROM and the memory 22.

The communication unit 23 is a communication circuit to connect to the communication network 3 wirelessly or by cable. The terminal 2 exchanges information via the communication unit 23 with the server 1 connected to the communication network 3.

The operation unit 25, which includes input operation controls, such as an operation button, a keyboard, and a touch panel, to provide various instructions, accepts an operation performed by a user and sends a signal corresponding to the operation to the controller 21. The terminal 2 may be constituted without the operation unit 25.

The display 24 includes a display screen, such as a liquid crystal display, and displays an image in accordance with control by the controller 21. A transparent touch panel of the operation unit 25 may be superimposed on the display screen.

The memory 22 is a high-capacity memory, such as a solid-state drive or a hard disk drive, and stores various programs, data, and the like that are read by the CPU in the controller 21.

The output unit 26 is a device that outputs in accordance with control by the controller 21 information to be perceived by a user through a sense other than sight (a sense of hearing, smell, touch, or the like). Examples of the output unit 26 include a loudspeaker that emits sound represented by audio data, an aroma emitter that evaporates an aromatic liquid to emit aroma in a space surrounding a user, a fan that produces an airflow and mist-like water droplets or the like accompanying the airflow, and a vibration generator that is in contact with a user and that transmits vibration to the user.

### Configuration of Server

FIG. 4 depicts a configuration of the server 1. The server 1 includes a controller 11, a memory 12, and a communication unit 13.

The controller 11 includes a CPU, a ROM, and a RAM. The CPU reads and executes programs to control each unit in the server 1, and these programs are stored in the ROM and the memory 12.

The communication unit 13 is a communication circuit to connect to the communication network 3 wirelessly or by cable. The server 1 exchanges information via the communication unit 13 with the terminal 2 and the information collecting apparatus 4 that are connected to the communication network 3.

The memory 12 is a high-capacity memory, such as a hard disk drive, and stores various programs that are read by the CPU in the controller 11.

The memory 12 stores an attendee database (DB) 121, a meeting DB 122, a history DB 123, and a mode DB 124.

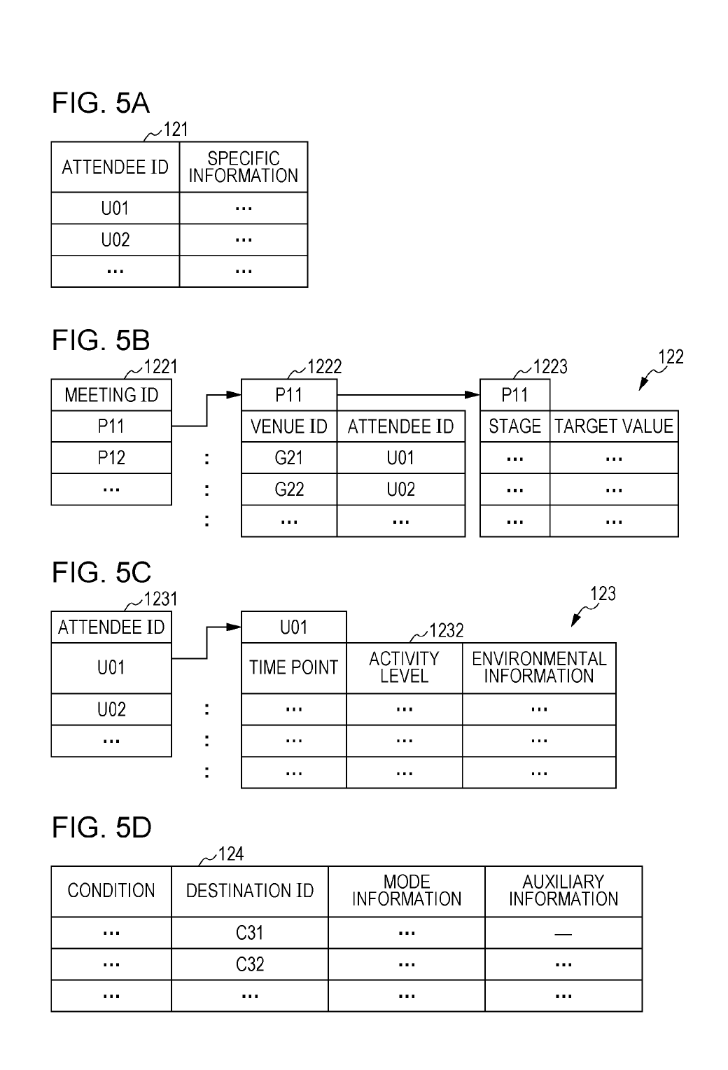

FIGS. 5A, 5B, 5C, and 5D depict databases stored in the memory 12. The attendee DB 121 is a database that stores information concerning the attendees attending a meeting. The attendee DB 121 depicted in FIG. 5A stores attendee identifiers (ID), each of which is identifying information to identify an attendee, along with pieces of specific information, and each piece of specific information is unique to an attendee and is associated with an attendee ID.

Examples of the specific information associated with an attendee include information used to normalize the activity level of the attendee and information that enables other people to recognize the attendee. Normalizing the activity level mentioned here indicates processing to express the activity level of the attendee as a ratio to compensate for variations among individuals by using the maximum and the minimum of the activity level that have been determined for the attendee in the past. The information that enables other people to recognize the attendee is information that is presented by the terminal 2 to enable other people to recognize the presence of the attendee, and examples of such information include theme music and a theme color that are associated with the attendee and a name, a likeness, and an icon of the attendee.

The meeting DB 122 is a database that stores information concerning attendees who attend meetings and target values for the activity level that attendees are to achieve in the meetings. The meeting DB 122 depicted in FIG. 5B includes a meeting list 1221, a venue list 1222, and a target value list 1223.

The meeting list 1221 is a list of meeting IDs, each of which is a piece of information that identifies a meeting. For each meeting ID, the venue list 1222 stores attendee IDs, each of which identifies an attendee in the meeting identified by the meeting ID, along with venue IDs, each of which is a piece of information to identify a venue. Each of the attendee IDs is associated with a venue ID, which identifies a venue where the attendee identified by the attendee ID is actually present.

For each meeting ID, the target value list 1223 stores stages in the progression of the meeting identified by the meeting ID along with target values for the activity level, and each stage is associated with a target value that the activity level of attendees is to reach in the stage. A facilitator who leads the meeting, such as the chairperson, an advisory member, or a presenter, may operate their own terminal 2 to set a stage in the progression of the meeting. The target value list 1223 may determine a target value in association with a period that has elapsed after the meeting begins or the time itself instead of a stage in the progression of the meeting.

A target value may be a single value or a group of values that indicates an interval defined by the upper limit and the lower limit. A target value is not limited to be relatively high and may be low. This is because, for example, attendees are desirably highly focused in some stage in the progression of the meeting but desirably calm and relaxed in another stage.

The target value may be determined for each attendee or for each venue. Further, the target value may be determined for each meeting (that is, for all the attendees in one meeting). If meetings are classified in accordance with the kind of meeting, the target value may be determined for each kind of meeting.

When a target value is determined for attendees who are in each venue or for all the attendees in a meeting, the target value may be a value that a statistical value of activity levels calculated for a plurality of attendees is to reach, or a value that the derivative or the integral of the statistical value thus calculated is to reach. Examples of such a statistical value calculated for the attendees include the maximum, the minimum, the median, the mode, the mean, the variance, the standard deviation, the range, the skewness, and the kurtosis.

The history DB 123 is a database to store how the activity level of each attendee in a meeting has changed. The history DB 123 depicted in FIG. 5C includes an attendee list 1231 and a history list 1232. The attendee list 1231 is a list of attendee IDs.

For each attendee ID included in the attendee list 1231, the history list 1232 stores a change, with passage of time, in the activity level and in the environmental information concerning an attendee identified by the attendee ID. For each time point, the history list 1232 stores the activity level determined at the time point for the attendee in association with the time point. The history list 1232 depicted in FIG. 5C also stores the environmental information determined at the time point for the attendee in association with the time point. In summary, the history list 1232 depicted in FIG. 5C stores a history of a combination of the activity level determined for an attendee and the environmental information determined for the attendee at the time point when the activity level is determined.

The mode DB 124 is a database that stores modes in which an attendee is to be represented in accordance with the activity level of the⁻attendee. The mode DB 124 depicted in FIG. 5D stores conditions concerning the activity level in association with pieces of mode information, and each piece of mode information indicates the mode in which an attendee is to be represented when the associated condition is satisfied.

Examples of a piece of mode information stored in the mode DB 124 include a ratio of an area representing an attendee. In such a case, for example, the mode DB 124 may determine the ratio of an area representing an attendee in a manner such that the ratio increases as the activity level increases or the ratio increases as the activity level decreases. In addition, the mode DB 124 may compare the activity level with a threshold and select attendees to be displayed on the terminal 2.

The mode DB 124 depicted in FIG. 5D also stores pieces of information, each of which identifies a destination (destination ID), in association with pieces of mode information. Each piece of mode information is associated with a destination to which information concerning an attendee represented in the mode indicated by the piece of mode information is transmitted. The destination identified by the destination ID may be the terminal 2 disposed at the venue where the attendee indicated by the information to be transmitted is present or the terminal 2 disposed at a venue other than the venue where the attendee is present. The destination identified by the destination ID may also be the terminal 2 to be checked by the facilitator of a meeting.

The mode DB 124 depicted in FIG. 5D also stores pieces of auxiliary information to assist in the progression of a meeting. The range of possible values for the difference between the activity level and the target value for the activity level is divided into successive intervals. When the activity level does not reach the target value for the activity level, a piece of auxiliary information is associated with a condition determined in accordance with the interval to which the difference between the activity level and the target value belongs. Examples of auxiliary information include a guidance message such as “The mean activity level appears lower than the target value. Take a rest.” or “One of the attendees does not seem to understand the contents. Do not proceed to the next stage. Stay in the present stage.”, and such a guidance message is transmitted, for example, to the terminal 2, which is a predetermined destination, of a meeting member such as the facilitator. If no auxiliary information is available, a piece of data indicating that no information is available (“−” in FIG. 5D) is stored under the title of the auxiliary information in the mode DB 124 depicted in FIG. 5D. Functional Configuration of Server

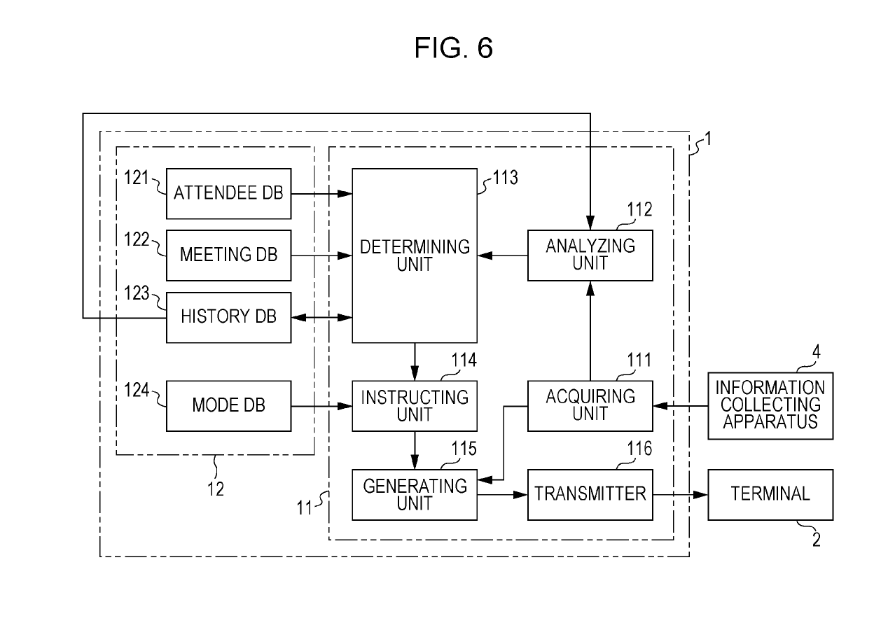

FIG. 6 depicts a functional configuration of the server 1. The controller 11 in the server 1 reads and executes programs, which are stored in the memory 12, to function as an acquiring unit 111, an analyzing unit 112, a determining unit 113, an instructing unit 114, a generating unit 115, and a transmitter 116. In FIG. 6, the communication network 3 and the communication unit 13 are not depicted.

The acquiring unit 111 acquires from the information collecting apparatus 4 image data representing a captured image of the attendee U and biological information of the attendee U. The biological information mentioned here is an electrocardiographic waveform that is measured by using electrodes of the measuring apparatus 5, which are attached to the attendee U, and that is sent to the server 1 by the information collecting apparatus 4. The image represented by this image data is an image that is captured by the imaging apparatus 7 and that is sent to the server 1 by the information collecting apparatus 4.

Talk by the attendee U and the sound originating in the area surrounding the attendee U may be captured, and the acquiring unit 111 may acquire audio data representing the captured sound from the information collecting apparatus 4.

In addition, the acquiring unit 111 may acquire environmental information including temperature, humidity, wind direction, wind speed, odor, and vibration in the area surrounding the attendee U. In short, the acquiring unit 111 may acquire environmental information obtained from the environment surrounding the attendee U.

The analyzing unit 112 analyzes the acquired biological information to determine the activity level of the attendee U. For example, the analyzing unit 112 analyzes the electrocardiographic waveform, which is one piece of the acquired biological information, in the frequency domain to obtain frequency components of the heartbeat, such as very low frequency (VLF), low frequency (LF), and high frequency (HF) components.

The term LF, which represents low frequency, indicates a variation wave signal originating from a blood pressure change (variation in blood pressure) that is referred to as a Mayer wave and that has a cycle period of approximately ten seconds. Alternatively, LF may indicate the sum (integral) of the power spectrum of the variation wave signal in the relevant frequency range. The frequency range relevant to LF is, for example, equal to 0.05 Hz or higher and lower than 0.15 Hz.

The term HF, which represents high frequency, indicates a variation wave signal originating from breath (variation in breath), which has a cycle period of approximately three to four seconds. Alternatively, HF may indicate the sum (integral) of the power spectrum of the variation wave signal in the relevant frequency range. The frequency range relevant to HF is, for example, equal to 0.15 Hz or higher and lower than 0.40 Hz.

Autonomic nerves are classified into sympathetic nerves and parasympathetic nerves. When the sympathetic nerves are excited, noradrenaline is emitted, and β receptors in cells located at a sinoatrial node receive the noradrenaline to start a chain of chemical reactions in the cells, increasing the heart rate.

When the parasympathetic nerves are excited, acetylcholine is emitted, and muscarine receptors in cells located at a sinoatrial node receive the acetylcholine to start chemical reactions in the cells, decreasing the heart rate.

The chemical reactions relating to the sympathetic nerves and to the parasympathetic nerves both have a recycling loop mechanism of chemical substances, and the substances used for the reactions are recycled and return to the original state. The reaction time for the substances to return to the original state is different for the sympathetic nerves and for the parasympathetic nerves, and thus a response time needed to respond to a received signal is different for the sympathetic nerves and for the parasympathetic nerves.

For example, the chemical reactions that relate to the sympathetic nerves and involve noradrenaline and β receptors are difficult to respond to a change with a cycle period less than approximately six seconds. Thus, the sympathetic nerves are difficult to transmit a variation in breath, which has a cycle period of approximately three to four seconds.

In contrast, the chemical reactions that relate to the parasympathetic nerves and involve acetylcholine and muscarine receptors proceed faster than the chemical reactions relating to the sympathetic nerves and thus are possible to respond to a change with a cycle period of approximately one second. Thus, the parasympathetic nerves are able to transmit a variation in breath.

A variation in blood pressure has a cycle period of approximately ten seconds, and thus the sympathetic nerves and the parasympathetic nerves are both able to transmit a variation in blood pressure.

When the sympathetic nerves are inhibited and the parasympathetic nerves are enhanced (stressed), a variation in blood pressure and a variation in breath are both reflected in a variation in heartbeat via the parasympathetic nerves. In contrast, when the sympathetic nerves are enhanced and the parasympathetic nerves are inhibited, a variation in blood pressure is reflected in a variation in heartbeat via the sympathetic nerves, but a variation in breath is not reflected in a variation in heartbeat.

Therefore, the HF component that reflects a variation in breath appears in a variation in heartbeat only when the parasympathetic nerves are activated (excited). In contrast, the LF component that reflects a variation in blood pressure appears in a variation in heartbeat when the sympathetic nerves are activated or the parasympathetic nerves are activated. Thus, when the HF component is large relative to the LF component, the parasympathetic nerves are found to be growing tense, and when the LF component is large relative to the HF component, the sympathetic nerves are found to be growing tense. For example, the LF component divided by the HF component (LF/HF) provides the activity of the sympathetic nerves and is treated as an index representing a stress level.

The analyzing unit 112 uses the high-speed Fourier transform, the Fourier-series expansion with unequal sampling intervals, the wavelet transform, or the like to analyze data. The analyzing unit 112 may transform a time series of an electrocardiographic waveform by using, for example, the third-order spline interpolation so that the R-R intervals become fixed. If the acquiring unit 111 acquires biological information other than an electrocardiographic waveform, such as a pulse wave or heart sounds of attendees, the analyzing unit 112 may analyze such biological information.

The determining unit 113 determines the activity level of the attendee U in accordance with the result of an analysis performed by the analyzing unit 112. Specifically, the determining unit 113 determines the activity level of the attendee U who attends a meeting in accordance with an analysis based on biological information acquired from the body of the attendee U.

The activity level of the attendee U is an index representing a degree to which the attendee U is activated and represents the degree of concentration, the depth of understanding, the willingness to participate in the meeting, and the like of the attendee U. The activity level is derived as a function of values that are calculated by the determining unit 113 (for example, the LF component and the HF component described above) by using the frequency-domain analysis, performed by the analyzing unit 112, of an electrocardiographic waveform. Examples of the activity level include the sum of the LF component and the HF component. The determining unit 113 may refer to the attendee DB 121 and normalize the activity level for each attendee.

The instructing unit 114 refers to the mode DB 124 and, if the activity level determined by the determining unit 113 for an attendee satisfies a predetermined condition, extracts from the mode DB 124 a piece of mode information indicating a mode in which the attendee is to be represented. Then, the instructing unit 114 provides the generating unit 115 with instructions on the mode indicated by the extracted piece of mode information. In summary, the instructing unit 114 provides instructions on a mode in which an attendee is to be represented in accordance with the activity level of the attendee.

For example, if the terminal 2 displays an image of an attendee by using the display 24 to represent the attendee, the instructing unit 114 may provide instructions on a mode in which the image of the attendee is to be displayed on the screen in accordance with the activity level of the attendee.

If conditions concerning a relationship between the activity level and the target value for the activity level are stored in the mode DB 124, the instructing unit 114 may provide instructions on a mode in which an attendee is to be represented in accordance with the activity level of the attendee and the target value for the activity level. In such a case, the instructing unit 114 may use the activity level and the target value for the activity level to extract the mode information described above from the mode DB 124 and may provide instructions on the mode. The activity level is determined by the determining unit 113, and the target value for the activity level is stored in the meeting DB 122.

When the activity level does not reach the target value, the instructing unit 114 may provide instructions to transmit to a predetermined destination auxiliary information to assist in the progression of a meeting in accordance with the difference between the activity level and the target value.

When the acquiring unit 111 acquires the environmental information described above, the instructing unit 114 may provide instructions on a mode in which an attendee is to be represented in accordance with the correlation between the activity level of the attendee and the environmental information. In such a case, the analyzing unit 112 refers to the history DB 123 and calculates a correlation coefficient between the activity level of the attendee and the environmental information. The instructing unit 114 may determine a mode to be provided in accordance with the calculated correlation coefficient. If a plurality of pieces of environmental information are present, the analyzing unit 112 may calculate a correlation coefficient individually for a combination of each piece of the environmental information and the activity level of the attendee.

The generating unit 115 generates information concerning an attendee to be represented in the mode provided by the instructing unit 114. For example, the generating unit 115 may receive from the acquiring unit 111 image data that represents an image of the attendee U, the image being captured by the imaging apparatus 7, and that is sent from the information collecting apparatus 4 to the server 1 and may process the image data in accordance with the mode provided by the instructing unit 114. In addition, the generating unit 115 may receive from the instructing unit 114 specific information concerning the attendee U, which is stored in the attendee DB 121 and read by the determining unit 113, and may process the specific information in accordance with the provided mode.

The transmitter 116 transmits to the terminal 2 information that concerns the attendee U and that is generated by the generating unit 115. In summary, the transmitter 116 transmits the information concerning the attendee U, who is represented in the provided mode.

If the instructing unit 114 provides instructions to transmit the auxiliary information described above, the transmitter 116 may transmit the auxiliary information to the provided destination.

### Server Operation

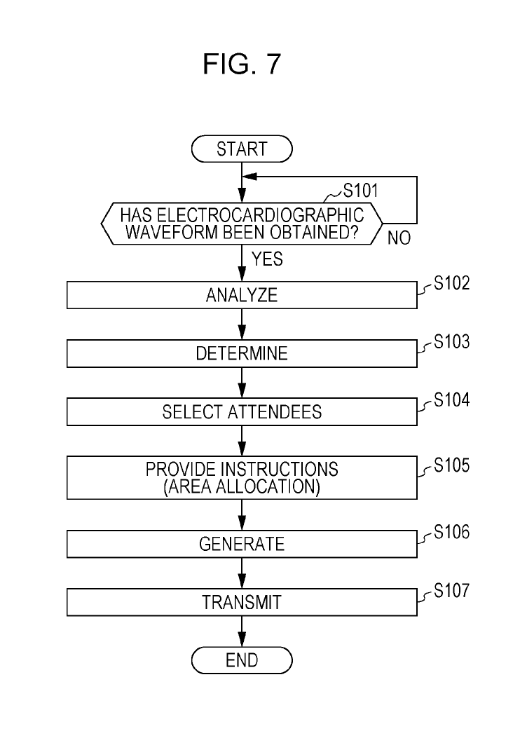

FIG. 7 is a flowchart depicting an operation flow performed by the server 1.

The controller 11 of the server 1 determines whether an electrocardiographic waveform of the attendee U (example biological information) has been obtained from the information collecting apparatus 4 (step S101) and repeats this processing while it is determined that no electrocardiographic waveform has been obtained (NO in step S101). When it is determined that an electrocardiographic waveform of the attendee U has been obtained (YES in step S101), the controller 11 analyzes the electrocardiographic waveform (step S102).

The controller 11 determines the activity level of the attendee U in accordance with the result of the analysis of the electrocardiographic waveform (step S103). Then, as a mode in which attendees are to be represented, the controller 11 selects attendees to be displayed on the terminal 2 in accordance with the activity levels of the attendees (step S104) and provides instructions to allocate an area for display to each of the selected attendees (step S105).

The controller 11 determines the layout of the screen in accordance with the ratio of the area allocated to each attendee and generates an image that combines captured images of the attendees (step S106). Each of the captured images of the attendees occupies an area proportional to the ratio of the area described above in the combined image. Then, the controller 11 transmits image data representing the generated image to the terminal 2 (step S107).

The terminal 2 receives the image data from the server 1 and causes the display 24 to display an image represented by the image data.

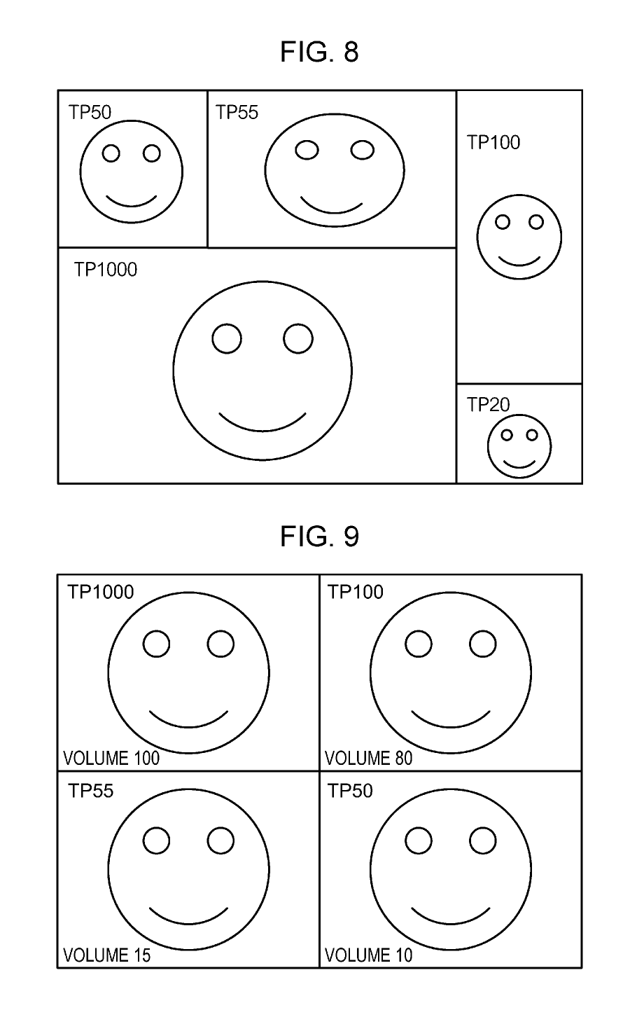

FIG. 8 depicts an example image displayed on the display 24 of the terminal 2. As depicted in FIG. 8, the display 24 of the terminal 2 displays activity levels of attendees (hereinafter, also referred to as TP) in a meeting along with images of the attendees. A portion in which an image of each attendee is displayed has an area based on the activity level of the attendee. For example, in the example depicted in FIG. 8, the largest portion in the screen is allocated for displaying the attendee having a TP of 1000, who has the highest TP among the attendees.

This screen is shared by the attendees in the meeting, and even an attendee at a remote location will know, among the attendees, who is concentrated, who is not concentrated, who is actively participating, and the like. In addition, the facilitator of the meeting may use this screen to determine on the progression of the meeting, such as whether to proceed to the next stage, who is to be prompted to speak, or the like. When displaying the attendees on the display 24, the controller 21 of the terminal 2 may prioritize an attendee at a remote location over an attendee at the venue where the terminal 2 is disposed if the two attendees have similar activity levels.

### Modifications

The exemplary embodiment has been described, and the exemplary embodiment may be modified as follows. Any combination of the following modifications is also feasible.

### Modification 1

In the exemplary embodiment described above, the mode DB 124 includes conditions concerning a relationship between the activity level and the target value for the activity level, but the conditions may be constituted without a portion relating to the target value. In such a case, the meeting DB 122 may be constituted without the target value list 1223.

### Modification 2

In the exemplary embodiment described above, when an activity level does not reach the target value for the activity level, a piece of auxiliary information is associated with a condition determined in accordance with the interval to which the difference between the activity level and the target value belongs, and the mode DB 124 stores the condition and the piece of auxiliary information to assist in the progression of a meeting. However, the mode DB 124 may be constituted without the auxiliary information.

### Modification 3

In the exemplary embodiment described above, the information collecting apparatus 4 provides the server 1 with the environmental information concerning an attendee, which is observed by the observing apparatus 6. But the information collecting apparatus 4 may avoid providing the environmental information. In such a case, the analyzing unit 112 need not calculate a correlation coefficient between the activity level of an attendee and the environmental information.

### Modification 4

In the exemplary embodiment described above, the mode DB 124 determines a ratio of an area representing an attendee in a manner such that the ratio increases as the activity level increases, but this is not limiting. For example, the mode DB 124 may determine the position of an image of an attendee in a manner such that an image of an attendee is disposed at a position closer to a predetermined position in the screen as the activity level of the attendee increases. Representation modes stored in the mode DB 124 may include background, luminance, chroma, and color, which are determined so as to vary in accordance with the activity level of an attendee.

In the exemplary embodiment described above, the terminal 2 displays an image of an attendee by using the display 24 to represent the attendee, but an attendee may be represented by using other methods. The terminal 2 may represent an attendee by using the output unit 26 in accordance with information transmitted by the server 1.

For example, if the output unit 26 is a loudspeaker, the output unit 26 may output a voice of an attendee, the sound in the surroundings of an attendee, or theme music predetermined by the information unique to an attendee. In such a case, the volume may be determined in accordance with the activity level of the attendee. Further in such a case, the ratio of the volume of the voice of an attendee to the volume of the sound in the surroundings of the attendee may be determined in accordance with the activity level of the attendee. Furthermore, in such a case, parameters used for processing the sound described above (for example, reverb, filtering, and the like) may be determined in accordance with the activity level of the attendee.

FIG. 9 depicts an example image displayed on the display 24 of the terminal 2 in this modification. In the example depicted in FIG. 9, although portions in which images of attendees are displayed all have the same area, mode information is determined in a manner such that an image of an attendee who has a higher activity level is disposed in the upper part of the screen and on the left-hand side of the screen. Thus, images of attendees are arranged from the top to the bottom in a column in descending order of activity level and from left to right in a row in descending order of activity level.

The terminal 2 that displays the image depicted in FIG. 9 controls the output unit 26 to increase the volume of the voice of an attendee as the activity level of the attendee increases or decreases. In such a configuration, for example, the voice of an attendee sounds louder as the attendee is more concentrated in a meeting.

The method to represent an attendee is not limited to a method to appeal to the sense of sight or hearing. For example, if the output unit 26 is an aroma emitter, the output unit 26 may emit aroma mixed in a ratio predetermined by the information unique to an attendee.

### Modification 5

In the exemplary embodiment described above, the instructing unit 114 provides instructions on a mode in which an attendee is to be represented in accordance with the activity level of the attendee. Instead, the instructing unit 114 may provide instructions on a mode in which a group including the attendee is to be represented in accordance with the activity levels totaled for the group including the attendee. Specifically, for example, for each venue, the instructing unit 114 may total the activity levels of attendees who are present at the venue to attend a meeting and may provide instructions on a mode in which the venue is to be represented in accordance with a statistical value, such as the mean, obtained as a result of totaling.

In such a case, the mode DB 124 may store a condition concerning the statistical value of the activity levels of the attendees present at the venue in association with a piece of mode information indicating the mode in which the venue is to be represented when the condition is satisfied. Then, in such a case, the transmitter 116 may transmit information on the venue represented in the provided mode.

In such a configuration, for example, the terminal 2 displays a scene at a venue for which the activity level exceeds a predetermined threshold, and thus attendees who watch the scene feel concentration or excitement at the venue and are stimulated.

### Modification 6

The transmitter 116 may transmit information when a predetermined period has elapsed since the preceding transmission. For example, if a condition stored in the mode DB 124 includes a threshold defined for the moving average of the activity levels averaged over this predetermined period, the instructing unit 114 provides instructions at intervals equal to this predetermined period, and thus the transmitter 116 transmits information at intervals equal to this predetermined period. In addition, the transmitter 116 may delay transmission until this predetermined period has elapsed even if information generated immediately after the preceding transmission is present.

Such a configuration enables the server 1 to set transmission intervals, and thus intervals at which the display of attendees on the terminal 2 is switched are controlled.

### Modification 7

The mode DB 124 may store a condition concerning the probability of change in activity level caused by the transmission, by the server 1, of information concerning an attendee. For example, the mode DB 124 may be devised in a manner such that, when the activity level of an attendee changes to a value exceeding a threshold after the server 1 transmits the first information concerning the attendee, information concerning the attendee is represented in a mode based on the degree of change. In such a case, the instructing unit 114 realized by the controller 11 may provide instructions to transmit information concerning an attendee, among the attendees, whose activity level changes to a value exceeding the threshold after the first information concerning the attendee is transmitted.

In such a configuration, since the server 1 transmits the information to the terminal 2 and the terminal 2 displays an attendee in accordance with the information, the attendee who has been affected to a degree exceeding the threshold is identified.

### Modification 8

In the exemplary embodiment described above, when the acquiring unit 111 acquires environmental information, the analyzing unit 112 calculates a correlation coefficient between the activity level and the environmental information, and the instructing unit 114 determines a mode to be provided in accordance with the calculated correlation coefficient. However, the instructing unit 114 may provide instructions to transmit the environmental information determined in accordance with the history of the activity level and the history of the environmental information.

For example, when the analyzing unit 112 performs an analysis by using the history of the activity level and the history of the environmental information and the determining unit 113 uses the analysis result to identify the environmental information shared by the attendees whose activity levels satisfy a predetermined condition, the instructing unit 114 may provide instructions to transmit the identified environmental information.

Such a configuration enables the server 1 to transmit the environmental information representing the environment shared by the attendees whose activity levels are found to satisfy a predetermined condition. The transmitted environmental information is used to study the environment to be provided at a venue so that the activity levels of the attendees satisfy the predetermined condition. Such a configuration enables the facilitator and the like of a meeting to grasp, for example, that the environmental condition at a venue for which the activity levels of attendees satisfy the predetermined condition is a temperature of 25° C. plus or minus 2° C. and a humidity level of 50% plus or minus 5% and that coffee is available for the attendees at such a venue. Modification 9

In the exemplary embodiment described above, the controller 11 reads and executes programs, which are stored in the memory 12, to function as the acquiring unit 111, the analyzing unit 112, the determining unit 113, the instructing unit 114, the generating unit 115, and the transmitter 116. However, some of these functions may be performed by the terminal 2 or the information collecting apparatus 4.

For example, the controller 41 in the information collecting apparatus 4 may realize a function equivalent to the function of the acquiring unit 111 described above when biological information is acquired from the measuring apparatus 5.

In addition, the controller 41 in the information collecting apparatus 4 may analyze the acquired biological information to determine the activity level of an attendee. In other words, the controller 41 may realize a function equivalent to the function of the analyzing unit 112 described above. In such a case, the controller 41 may transmit information indicating the analysis result to the server 1, and the controller 11 in the server 1 may determine the activity level of the attendee in accordance with the transmitted information.

Furthermore, for example, the controller 21 in the terminal 2 may receive from the server 1 mode information indicating the mode in which an attendee is to be represented and may generate information to represent the attendee in the mode indicated by the received mode information. In other words, the controller 21 may realize a function equivalent to the function of the generating unit 115 described above.

In summary, the information processing system 9 in this modification includes the information collecting apparatus 4 that includes an acquiring unit that acquires biological information from a body of each attendee attending a meeting, the terminal 2 that displays an attendee in accordance with generated information concerning the attendee, and the server 1 that communicates with the information collecting apparatus 4 and the terminal 2.

The information collecting apparatus 4 or the server 1 in the information processing system 9 includes an analyzing unit that analyzes the activity level of an attendee in accordance with the biological information.

The server 1 in the information processing system 9 includes an instructing unit that provides instructions on a mode in which an attendee is to be represented in accordance with the analyzed activity level.

The server 1 or the terminal 2 in the information processing system 9 includes a generating unit that generates information concerning the attendee represented in the provided mode.

In the information processing system 9, the terminal 2 may also have the function of the information collecting apparatus 4.

### Modification 10

In the exemplary embodiment described above, the transmitter 116 transmits to the terminal 2 information that is generated by the generating unit 115 and that concerns attendees. In addition to this, or instead of this, the transmitter 116 may transmit to the information collecting apparatus 4 a control signal used for acquiring information concerning an attendee.

For example, the transmitter 116 may transmit to the information collecting apparatus 4 a control signal to control the focal length of an optical system that captures an image of an attendee in accordance with the activity level of the attendee. In such a case, the information collecting apparatus 4 sends the received control signal to the imaging apparatus 7, and the imaging apparatus 7 performs control in accordance with the control signal.

In addition, the transmitter 116 may transmit to the information collecting apparatus 4 a control signal to control the directivity in capturing a voice of an attendee in accordance with the activity level of the attendee. In such a case, the information collecting apparatus 4 sends the received control signal to the sound-capturing apparatus 8, and the sound-capturing apparatus 8 performs control in accordance with the control signal.

### Modification 11

Programs to be executed by the controller 11 in the server 1 may be recorded on a computer-readable recording medium and provided. Examples of such a computer-readable recording medium include a magnetic recording medium, such as a magnetic tape or a magnetic disk, an optical recording medium, such as an optical disc, a magneto-optical recording medium, and a semiconductor memory. These programs may be downloaded via a communication network, such as the Internet. Various devices other than a CPU may realize a controller illustrated by the controller 11 described above. For example, a specific-purpose processor or the like may be used.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a determining unit that determines an activity level of an attendee attending a meeting in accordance with an analysis based on biological information acquired from a body of the attendee;
an instructing unit that provides instructions on a mode in which the attendee is to be represented in accordance with the activity level;
a generating unit that generates information concerning the attendee represented in the mode; and
a transmitter that transmits the generated information concerning the attendee.

2. The information processing apparatus according to claim 1,
wherein the instructing unit provides instructions on the mode in accordance with the activity level and a target value for the activity level.

3. The information processing apparatus according to claim 2,
wherein, when the activity level does not reach the target value, the instructing unit provides instructions to transmit to a predetermined destination auxiliary information to assist in progression of the meeting in accordance with a difference between the activity level and the target value, and
the transmitter transmits the auxiliary information to the provided destination.

4. The information processing apparatus according to claim 1, further comprising:
an acquiring unit that acquires environmental information obtained from an environment surrounding the attendee,
wherein the instructing unit provides instructions on the mode in accordance with a correlation between the activity level of the attendee and the environmental information.

5. The information processing apparatus according to claim 2, further comprising:
an acquiring unit that acquires environmental information obtained from an environment surrounding the attendee,
wherein the instructing unit provides instructions on the mode in accordance with a correlation between the activity level of the attendee and the environmental information.

6. The information processing apparatus according to claim 3, further comprising:
an acquiring unit that acquires environmental information obtained from an environment surrounding the attendee,
wherein the instructing unit provides instructions on the mode in accordance with a correlation between the activity level of the attendee and the environmental information.

7. The information processing apparatus according to claim 1,
wherein the instructing unit provides instructions on a mode in which an image of the attendee is displayed on a screen in accordance with the activity level.

8. The information processing apparatus according to claim 2,
wherein the instructing unit provides instructions on a mode in which an image of the attendee is displayed on a screen in accordance with the activity level.

9. The information processing apparatus according to claim 3,
wherein the instructing unit provides instructions on a mode in which an image of the attendee is displayed on a screen in accordance with the activity level.

10. The information processing apparatus according to claim 4,
wherein the instructing unit provides instructions on a mode in which an image of the attendee is displayed on a screen in accordance with the activity level.

11. The information processing apparatus according to claim 5,
wherein the instructing unit provides instructions on a mode in which an image of the attendee is displayed on a screen in accordance with the activity level.

12. The information processing apparatus according to claim 6,
wherein the instructing unit provides instructions on a mode in which an image of the attendee is displayed on a screen in accordance with the activity level.

13. The information processing apparatus according to claim 1,
wherein, the instructing unit provides instructions on a mode in which a group including the attendee is to be represented in accordance with the activity levels totaled for the group including the attendee, and
the transmitter transmits the information concerning the group represented in the mode.

14. The information processing apparatus according to claim 1,
wherein the transmitter transmits the generated information when a predetermined period has elapsed since preceding transmission.

15. The information processing apparatus according to claim 1,
wherein, if there is an attendee whose activity level changes to a level exceeding a threshold after the information concerning the attendee is transmitted, the instructing unit provides instructions to transmit information concerning the attendee.

16. The information processing apparatus according to claim 4, further comprising:
a memory that stores a history of a combination of the activity level determined for the attendee and the environmental information determined for the attendee at a time point when the activity level is determined,
wherein the determining unit identifies from the history the environmental information shared by the attendees whose activity levels satisfy a predetermined condition, and
the instructing unit provides instructions to transmit the identified environmental information.

17. The information processing apparatus according to claim 5, further comprising:
a memory that stores a history of a combination of the activity level determined for the attendee and the environmental information determined for the attendee at a time point when the activity level is determined,
wherein the determining unit identifies from the history the environmental information shared by the attendees whose activity levels satisfy a predetermined condition, and
the instructing unit provides instructions to transmit the identified environmental information.

18. The information processing apparatus according to claim 6, further comprising:
a memory that stores a history of a combination of the activity level determined for the attendee and the environmental information determined for the attendee at a time point when the activity level is determined,
wherein the determining unit identifies from the history the environmental information shared by the attendees whose activity levels satisfy a predetermined condition, and
the instructing unit provides instructions to transmit the identified environmental information.

19. An information processing system comprising:
an information collecting apparatus that includes an acquiring unit that acquires biological information from a body of each attendee attending a meeting;
a terminal that displays the attendee; and
an information processing apparatus that communicates with the information collecting apparatus and the terminal,
wherein the information collecting apparatus or the information processing apparatus includes
an analyzing unit that analyzes an activity level of the attendee in accordance with the biological information,
the information processing apparatus includes
an instructing unit that provides instructions on a mode in which the attendee is to be represented in accordance with the analyzed activity level,
the information processing apparatus or the terminal includes
a generating unit that generates information concerning the attendee represented in the mode, and
the terminal displays the attendee in accordance with the generated information concerning the attendee.

20. An information processing apparatus comprising:
means for determining an activity level of an attendee attending a meeting in accordance with an analysis based on biological information acquired from a body of the attendee;
means for providing instructions on a mode in which the attendee is to be represented in accordance with the activity level;
means for generating information concerning the attendee represented in the mode; and
means for transmitting the generated information concerning the attendee.
